(19) United States
(12) Patent Application Publication
(10) Pub. No.: US 2022/0096168 A1
(43) Pub. Date: Mar. 31, 2022

(54) Retroreflective Markers For A Three-Dimensional Tracking System

(71) Applicant: Northern Digital Inc., Waterloo (CA)

(72) Inventors: Shaulaine White, Cambridge (CA); Elsabe Coetzer, Waterloo (CA); Larry Chen, Fergus (CA); Athanasios Tommy Balkos, Waterloo (CA)

(21) Appl. No.: 17/485,081

(22) Filed: Sep. 24, 2021

Related U.S. Application Data
(60) Provisional application No. 63/083,501, filed on Sep. 25, 2020.

(51) Int. Cl. A61B 34/20; G06F 3/03; A61B 90/00

(52) U.S. Cl. CPC A61B 34/20; G06F 3/0308; A61B 90/39; A61B 2090/3991; A61B 2034/2057; A61B 2090/3979; A61B 2090/3987; A61B 2034/2065

(57) ABSTRACT

A marker for an optical tracking system includes an object having a curved surface and a plurality of beads adhered to the curved surface with an adhesive. The beads of the plurality of beads are configured for being retroreflectors. A bead of the plurality of beads can include a first portion having a reflective surface and a second portion that is substantially transparent. The first portion of the bead is oriented toward the curved surface of the object. The second portion of the bead is oriented away from the curved surface of the object. The beads are applied to the marker. A bead of the plurality of beads can be transparent and embedded in a reflective medium on the marker surface.

### CLAIM OF PRIORITY

This application claims priority under 35 U.S.C. § 119(e) to U.S. Patent Application Ser. No. 63/083,501, filed on Sep. 25, 2020, the entire contents of which are hereby incorporated by reference.

### TECHNICAL FIELD

This disclosure relates to an optical tracking system and marker.

### BACKGROUND

Tracking systems typically rely on objects having one or more markers affixed thereto. The markers that are affixed to the object may be active markers (e.g., light emitting diode markers), passive markers or a combination of active and passive markers.

Generally, passive markers can be configured to reflect an optical signal toward a camera. The marker can be configured to reflect the optical signal on a parallel path back toward the signal source. Based on when the camera detects an optical signal reflected from the marker, a tracking system can estimate a position of the marker in an environment.

In a medical application context, a user (e.g., a doctor) touches a surface of interest (e.g., a surface of a patient's body) using a distal tip of an object (e.g., a probe or a surgical instrument.) An object sensing device views the marker(s) affixed to the object. On the basis of the known locations of the sensing device and the location of the object(s) as seen by the sensing device, such systems calculate the three-dimensional coordinates of the object(s).

### SUMMARY

A tracking system is configured to determine a position of the marker in an environment. The marker includes a reflective element that provides a signal to indicate the position and orientation (e.g., pose) of the marker in the environment. Generally, the tracking system can be an optical tracking system, and the marker is a passive markers configured to reflect an optical signal. The tracked object can include a retroreflective coating configured to reflect an optical signal along a parallel path back towards a source of the optical signal. Generally, an optical sensor (e.g., a camera) is positioned near the source of the optical signal and configured to detect the reflected optical signal from the marker. A reflection (e.g., an illumination, glint, etc.) is detected on the marker. The tracking system is configured to estimate where the marker is in the environment based on where in the image the reflected signal is detected.

The marker generally includes a substrate material with a surface that is coated with retroreflective beads. The beads are small relative to the size of the marker and can be made of glass or a similar material. Generally, the marker is spherical, though other geometries are possible. The beads are deposited on the surface of the marker as uniformly as possible. There are several different ways to cause the spherical surface marker to have uniform retro-reflectivity. In an example, a process includes partially coating the beads with a reflective conductor (e.g., aluminum or silver or an alloy thereof). The process includes orienting the beads along the spherical surface of the marker so that the uncoated portion (e.g., uncoated half) is facing away from the marker surface. The beads are adhered to the marker surface in this orientation. When incoming light travels through a bead of the beads, the light (e.g., visible light, infrared light, etc.) is reflected off the metallic coating and back along a nearly parallel return path due to a refractive index of the bead. In another example, the beads do not have a reflective coating. The beads are adhered to a marker surface using a medium that hardens to hold the beads in place. The medium includes a sufficient density of reflective or conductor particles to reflect enough incoming light off the boundary surface between the bead and the particulate in the medium. The light is reflected back only a nearly parallel return path.

The implementations described herein can provide various technical benefits.

A spherical form factor for markers is symmetrical from any direction. For this reason, spherical markers are generally used for passive three dimensional (3D) optical tracking targets. The processes for producing the markers described in this specification do not require a flat retroreflective material to be formed onto a 3D spherical surface. Generally, a flat retroreflective material is populated with a uniform density of microspheres (beads,) with a reflective backing, which consequently produce uniform levels of reflected brightness across the surface. When the flat retroreflective material is applied to a spherical or curved surface of a marker, the material can be stretched which can result in non-uniformities in bead density. The processes described in this specification result in uniform bead density on the spherical surface because the beads are applied directly to the spherical surface, rather than to a flat material applied to the spherical surface. This avoids stretching the reflective material or causing overlapping multiple smaller segments of material, which cause non-uniform bead density. Instead, the bead density is uniform or nearly uniform over the spherical surface. This results in a uniform reflective surface and a uniform brightness when the marker is illuminated by the illumination source. This reduces tracking errors for 3D tracking. The tracking errors can occur when using non-uniform reflective markers in multiple orientations, which most applications require. The markers and processes for coating the markers described in this document result in a marker with a uniform surface at any orientation.

In a general aspect, a marker for an optical tracking system includes an object having a curved surface. The marker includes a plurality of beads adhered to the curved surface with an adhesive, the beads of the plurality configured for being retroreflectors. A bead of the plurality of beads includes a first portion having a reflective surface and a second portion that is substantially transparent. The first portion of the bead is oriented toward the curved surface of the object. The second portion of the bead is oriented away from the curved surface of the object.

In some implementations, the bead of the plurality of beads further includes a bead body that is configured to be a dipole. A first pole of the dipole is positioned at the first portion of the bead. A second pole of the dipole is positioned at the second portion of the bead.

In some implementations, the bead comprises a polarizable material that is at least substantially transparent in a near infrared (NIR) band with refractive index that is greater than 1.6. In some implementations, the bead includes a material selected from a group of materials including Barium Titanate.

In some implementations, the object is a spheriod. In some implementations, the object a three dimensional object including a ruled surface. In some implementations, the object is one of a cone, a cube, or a cylinder.

In some implementations, the bead is a spheroid. The first portion is approximately a first hemisphere of the spheriod, and the second portion is a second hemisphere of the spheriod. In some implementations, the reflective surface comprises aluminum, silver, or a combination thereof.

In a general aspect, a process for producing a reflective marker for an optical tracking system includes obtaining a plurality of beads each having a reflective portion and a substantially clear portion. The process includes obtaining an object having a curved surface, the curved surface including an adhesive. The process includes placing the plurality of beads in a solution. The process includes placing the object in the solution. The process includes applying a first electric potential to the solution, wherein the electric potential orients the reflective portion of each of the plurality of beads toward the curved surface of the object. The process includes applying a second electric potential to the object to attract the beads of the plurality to the curved surface. The adhesive on the curved surface holds each of the beads on the curved surface of the object. The reflective portion of each bead is oriented toward the curved surface. The substantially clear portion is oriented away from the curved surface.

In some implementations, each bead further includes a bead body that is configured to be a dipole. A first pole of the dipole is positioned at the reflective portion of the bead. A second pole of the dipole is positioned at the substantially clear portion of the bead. In some implementations, the bead comprises a polarizable material that is at least substantially transparent in a near infrared (NIR) band with refractive index that is greater than 1.6. In some implementations, the bead includes a material selected from a group of materials including Barium Titanate.

In some implementations, the object is a spheriod. In some implementations, the object a three dimensional object including a ruled surface. In some implementations, the object is one of a cone, a cube, or a cylinder.

In some implementations, the bead is a spheroid. The reflective portion is approximately a first hemisphere of the spheroid. The substantially clear portion is a second hemisphere of the spheriod. In some implementations, reflective portion comprises aluminum, silver, an alloy of aluminum or silver, or a combination thereof.

In a general aspect, a process for producing a reflective marker for an optical tracking system includes obtaining a plurality of beads each being substantially transparent. The process includes obtaining an object having a curved surface, the curved surface including an adhesive, wherein the adhesive comprises reflective particles. The process includes applying the plurality of beads to the curved surface, wherein the beads are partially embedded in the adhesive.

In some implementations, applying the plurality of beads to the curved surface a low-pressure pneumatic application of the beads to the adhesive. In some implementations, the reflective particles in the adhesive comprise silver, aluminum, or a combination thereof.

In some implementations, a size of the reflective particles is approximately one-tenth or less of a size of the beads.

In some implementations, applying the plurality of beads to the curved surface comprises embedding about one half of each of the beads in the adhesive. In some implementations, applying the plurality of beads to the curved surface comprises applying an electric potential to the object. The electric potential attracts the beads to adhere to the adhesive.

The details of one or more embodiments are set forth in the accompanying drawings and the description below. Other features and advantages will be apparent from the description and drawings, and from the claims.

### BRIEF DESCRIPTION OF THE DRAWINGS

FIG. 1 is a block diagram of an example tracking system.

FIGS. 2A-2C are diagrams of markers.

FIG. 3A is a diagram of a reflective bead.

FIG. 3B is a diagram that illustrates applying the reflective bead of FIG. 3A to a marker surface.

FIG. 4A is a diagram of a reflective bead.

FIG. 4B is a diagram of that illustrates applying the reflective bead of FIG. 4A to a marker surface.

FIG. 5A is a diagram of a reflective bead.

FIG. 5B is a diagram of a process for applying the reflective bead of FIG. 5A to a marker surface.

FIGS. 6A-6B are images of a marker in a non-illuminated state and in an illuminated state.

FIG. 7A is a flow diagram showing an example process for coating a marker with a reflective material.

FIG. 7B is a flow diagram showing an example process for coating a marker with a reflective material.

FIG. 8 is a diagram of an example computing system.

Like reference numbers and designations in the various drawings indicate like elements.

### DETAILED DESCRIPTION

This specification describes a marker for a tracking system. For example, the marker comprises an optical tracking target for an optical tracking system, which is subsequently described in further detail. The marker generally includes a substrate or body that has a retro-reflective coating or material on the surface of the marker. A retro-reflective material is a material that reflects electromagnetic waves back to its source with a reduced amount of scattering occurring in other directions (e.g., minimal scattering). For example, an electromagnetic wave (e.g., of a light wave) is reflected back along a vector that is substantially parallel to but opposite in direction from the wave source. The use of retro-reflective material on the passive marker can ensure that only electromagnetic waves from an illuminator are substantially reflected back at the image sensor.

Generally, the marker includes uniformity of the reflective surface. The greater the uniformity of the surface of the marker, the greater the corresponding reduction in tracking errors caused by improperly reflected electromagnetic signals from the marker. The processes for coating the marker with a reflective surface described below result in a uniform or nearly uniform reflective surface that results from coating the surface of the marker with a reflective material. Generally, the marker is directly coated with the reflective material. Markers can have a curved surface (e.g., spherical markers, cylindrical markers, etc.). By directly applying the reflective coating to the marker surface, the marker surface is nearly uniformly reflective from any viewing angle. In contrast, when a flat material is first coated with the reflective coating and then applied to the curved marker surface, a non-uniform reflective surface may result from stretching or overlapping pieces of the flat reflective material on the curved surface.

The reflective coatings generally include retroreflective beads. The beads are small relative to the size of the marker. In some implementations, the beads are made of glass or a similar material that has an index of refraction that results in a retro-reflection of received light back to a light source from the bead (e.g., the average index>1.8). Generally, the marker geometry is spherical, though other geometries are possible. The beads are deposited on the surface of the marker as uniformly as possible. There are several different ways to cause the spherical surface marker to have uniform retro-reflectivity. In an example, a process includes partially coating the beads with a pure and reflective conductor (e.g., aluminum or silver, an alloy of silver or aluminum, or any combination thereof). The process includes orienting the beads along the spherical surface of the marker so that the uncoated portion (e.g., uncoated half) is facing away from the marker surface. The beads are adhered to the marker surface in this orientation. When incoming light travels through a bead of the beads, the light (e.g., visible light, infrared light, etc.) is reflected off the metallic coating and back along a nearly parallel return path due to a refractive index of the bead. In another example, the beads do not have a reflective coating. The beads are adhered to a marker surface using a medium that hardens to hold the beads in place. The medium includes a sufficient density of reflective or conductor particles to reflect enough incoming light off the boundary surface between the bead and the particulate in the medium. The light is reflected back only a nearly parallel return path.

Generally, there are several example processes for producing the reflective coating on the markers. In a first example, the beads are partially coated with a pure and reflective conductor such as aluminum or silver. The beads are oriented along the spherical surface of the marker, such that the uncoated half is facing away from the surface. The beads are adhered to the marker surface in this orientation. Incoming light travels through the bead, is reflected off the metallic coating, and back along a nearly parallel return path due to the index of the bead (e.g., the index value is generally greater than 1.8). In another example, the beads have no reflective coating. Rather the beads are adhered to the spherical surface using a medium that hardens to hold the beads in place on the marker surface. The medium has a sufficient density of reflective/conductor particles for enough incoming light to be reflected off the boundary surface between the glass bead and the particulate in the medium to illuminate the marker surface. Generally, light is reflected back only a nearly parallel return path. The retroreflective coatings and the processes for producing the reflective coatings are subsequently described in greater detail.

A tracking system is configured to determine a position of the marker in an environment. The marker includes a reflective element that provides a signal to indicate the position and orientation (e.g., pose) of the marker in the environment. Generally, the tracking system can be an optical tracking system, and the marker is a passive markers configured to reflect an optical signal. The tracked object can include a retroreflective coating configured to reflect an optical signal along a parallel path back towards a source of the optical signal. Generally, an optical sensor (e.g., a camera) is positioned near the source of the optical signal and configured to detect the reflected optical signal from the marker. A reflection (e.g., an illumination, glint, etc.) is detected on the marker. The tracking system is configured to estimate where the marker is in the environment based on where in the image the reflected signal is detected.

Generally, the image sensor generates one or more images of a measurement volume including the marker. The one or more images are analyzed to identify positions of the marker in the one or more images for which {U, V} or {row, column} image coordinates are calculated, often to sub-pixel resolution. These {U, V} coordinates from one or more image sensors are used to compute the three dimensional (3D) position of the marker in a predetermined coordinate system (e.g., a Cartesian system, a polar coordinate system, etc.). To make the image processing easier, the system can be designed so that the marker provides high contrast images in which the marker illuminated or bright relative to the rest of the image. The high contrast can be achieved using a retro-reflective material that reflects electromagnetic waves emitted from an illumination source around the image sensor

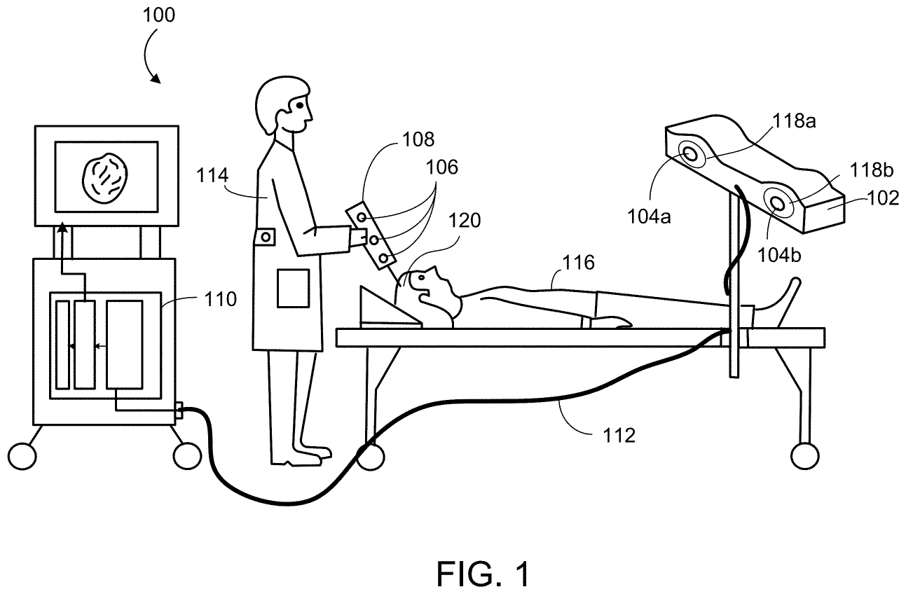

Referring to FIG. 1, a tracking system 100 includes an illumination/image capture unit 102 in which a marker sensing device (e.g., a camera, an array of cameras 104a-b, etc.) and marker illuminating device(s) 118a-b (e.g., electromagnetic waves source) that are rigidly mounted. In this example, the illuminating devices 118a-b emit electromagnetic waves, such as visible light, infrared light, etc. The electromagnetic waves are directed at a region that includes one or more retroreflective markers 106 that are affixed to an object. In the context shown in FIG. 1, the object can be a tool 108 (e.g., a surgical tool, medical device for treating a patient, etc.). The object can also be called a tracked object. The retroreflective markers 106 are configured to have retro-reflectivity to reflect incoming electromagnetic waves in a parallel and opposite direction from the incoming direction. The cameras 104a-b capture one or more images of the illuminated markers 106. Due to the highly retro-reflective nature of the markers 106, each marker appears as a relatively bright spot in the captured images, and the system can determine the spatial coordinates (e.g., Cartesian, spherical, cylindrical, etc.) and an intensity value that represents, for example, the brightness of each corresponding spot. This data is provided to a computing device (e.g., a processor) of a computing system 110. The computing device is configured to determine where in the region or environment the markers 106 are with respect to the cameras 104a-b.

Generally, the computing device is part of the computer system 110 that is connected to the array of cameras 104a-b via communication links 112 (e.g., wired communication links or wireless communication links). In other examples, the computing device is located within the camera mounting unit 102. The computing system 110 may include one or more of various forms of digital computers, including, e.g., laptops, desktops, workstations, personal digital assistants, servers, blade servers, mainframes, and other appropriate computers. The computing system 110 may include one or more of various forms of mobile devices, including, e.g., personal digital assistants, tablet computing devices, cellular telephones, smartphones, and other similar computing devices. The components shown here, their connections and relationships, and their functions, are meant to be examples only, and are not meant to limit implementations of the techniques described and/or claimed in this document.

Given the known locations of the cameras 104a-b included in the array and the locations of the retro-reflective markers 106, the computing device calculates a position and/or orientation of the object 108. Further, on the basis of the known relationship between the location of each of the retro-reflective markers 106 and the location of a tip 120 of the object 108 in the working volume (e.g., a tool coordinate system), the computing device calculates the coordinates of the tool tip 120 in space. In those instances in which the tool 108 is handled by a user (e.g., a surgeon 114) and the tool tip 120 is pressed against or is otherwise in contact with a surface (e.g., a body 116 of a patient), the coordinates of the tool tip 120 correspond to the coordinates of the point at which the tool tip 120 contacts the surface.

In some implementations, the reflective coatings for the markers 106 each include reflective beads comprising one or more of glass microspheres, plastic microprisms, etc. The reflective beads generally include a material for which the refractive index is configured to provide retro-reflection of light. Dyed plastics may also be used. In some implementations, barium titanate is used for the beads because of its crystalline structure, as subsequently described.

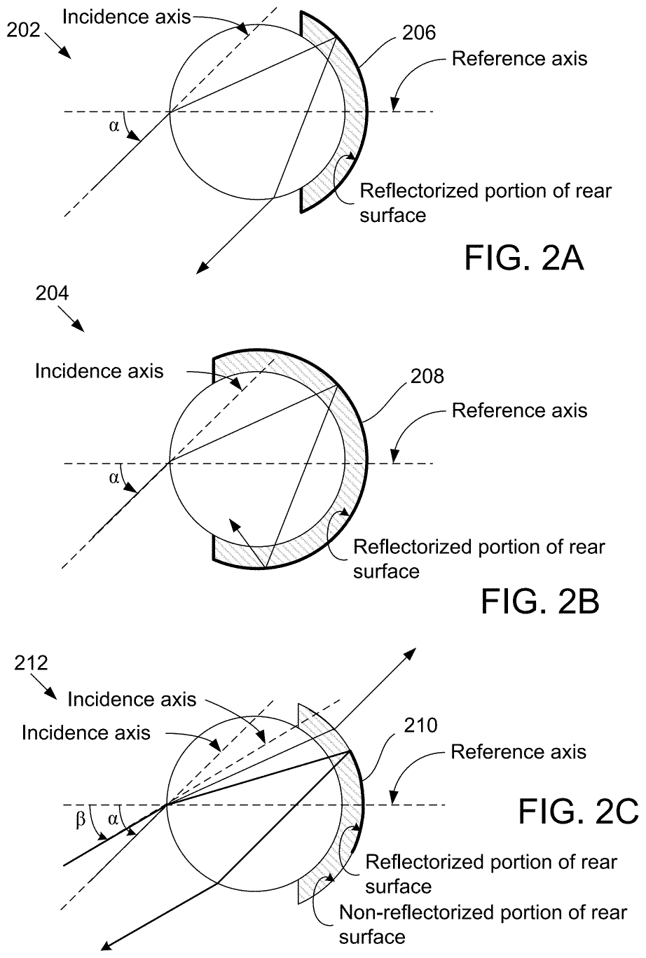

Turning to FIGS. 2A-2C, show examples of a retroreflective beads 202, 204 are shown that are microspheres that have respective caps 206, 208 attached to the rear surface of the microsphere. The examples of FIGS. 2A, 2B, and 2C illustrate the manner in which a retroreflective bead may be tuned (by varying the location of the cap and/or the size, geometry, etc. of the reflectorized rear surface) so that its retroreflective capability is “turned on” only when electromagnetic waves enters the bead within particular entrance angles. In some embodiments, the entirety of the rear surface of each of the beads 202, 204 is reflectorized and electromagnetic waves enters each bead (e.g. the beads 202 and/or 204) at an identical high entrance angle α. In the case of the bead 202, the shorter cap 206 enables an input electromagnetic waves ray to pass through the bead 202, reflect off the reflectorized rear surface, and exit the bead 202 as shown in FIG. 2A. By contrast, the more encompassing cap 208 causes multiple reflections of the incident ray and results in the reflected electromagnetic waves exiting the bead 204 at an exit angle that is not parallel to the angle of the input electromagnetic waves ray as shown in FIG. 2B. FIGS. 2A and 2C show examples of retroreflective beads 202, 212 having identically sized caps 206, 210 but different amounts of reflectorized rear surfaces. Suppose electromagnetic waves enters each of the beads 202, 212 at the high entrance angle α. In the case of the bead 212, the incoming electromagnetic waves passes through the bead 212, hits a non-reflectorized portion of the rear surface of the bead 212, and exits the bead as shown in FIG. 2C. A ray received at incident angle β will not reflect and just transmit through the portion of the cap that has a non-reflectorized portion rear surface.

The beads may be made of various materials, shapes, sizes, and materials for retro-reflection. For example, a material can be used that reflects infrared electromagnetic waves, UV electromagnetic waves, visible light, etc. In certain embodiments, the bead is made of solid glass, is approximately spherical in shape with diameter that is less than 200 micrometers (μm) (e.g., 20 μm-200 μm). The reflective coating can be a metallic coating, (e.g., a silver based coating, aluminum based coating, copper based coating, etc.) or a non-metallic thin film stack. The bead can take various shapes (e.g., sphere).

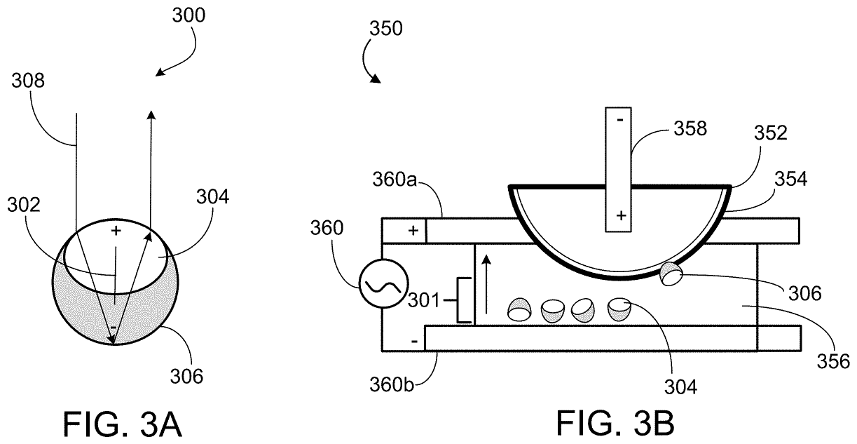

FIGS. 3A and 3B show examples of a bead 300 and a diagram of a process 350 for depositing the bead 300 on a curved surface of a marker 352. The bead 300 includes a sphere that is made of glass or similar material, as previously described. The bead 300 includes a reflective coating on a portion 306 of the surface of the bead. The reflective coating can include silver, aluminum, or any similar reflective material. In some implementations, barium titanate is used for the bead 300 as a clear material because it includes a crystalline structure for which a dipole can be maintained. The dipole is used to control orientation of the bead 300 and its reflective portion 306, as subsequently described.

The bead 300 is configured to have a dipole 302. The dipole 302 is based on a crystalline structure of the bead 300. The dipole 302 is aligned with the reflective portion 306 and non-reflective portion 304 (e.g., transparent or substantially transparent portion). In an example, the positive portion of the bead is non-reflective portion 304, while the negatively charged portion is a reflective portion 306. However, the polarity can generally be reversed such that reflective portion 306 is positively charged and non-reflective (transparent) portion 304 is negatively charged. Generally, the bead 300 has the same polarity as other beads to be applied to the surface of a marker. The coating on the reflective portion 306 is such that an electromagnetic signal 308 is reflected on a parallel but opposite vector to the incoming vector. This can be tuned as described previously in relation to FIGS. 2A-2C.

Generally, a substantially transparent or transparent portion is transmissive in the near infrared (NIR) band of the EM spectrum at a minimum, but can include additional bands. Because the refractive index that causes retro-reflectivity in the bead, the refractive index is generally greater than 1.6 in the NIR band.

Because the dipole of each bead 300 is aligned with the reflective and non-reflective portions of the bead as shown in FIG. 3A, the orientation of the bead can be controlled in a liquid using an electric potential applied to the beads. In this way, to coat a marker with the beads, the beads are aligned with each other so that all the reflective surfaces of the beads for the coating are facing in a particular orientation with respect to the marker. For example, the beads are aligned such that the beads have their respective reflective portions aligned towards a surface of the marker using an electric potential. The beads are not necessarily aligned with one another, but rather aligned with respect to the marker surface.

FIG. 3B shows a process 350 for coating a marker 352 with beads 301 (e.g., including bead 300). Generally, the marker 352 includes a substrate or body to which the beads 301 are adhered. The substrate or body can include most any material that is configured to be charged by the source of electric potential 358 to polarize the marker substrate. The beads 301 are placed in a solution 356, such as distilled water. The marker 352 is also at least partially submerged in the solution 356. The marker 352 is charged to a potential using the source of electric potential 358. Generally, the polarity of the potential of the marker 352 is opposite the polarity of the reflection portions of the beads 301. The reflective portions of the beads 301 are thus oriented towards the surface of the marker 352.

The solution 356 is charged with by a source 360 of electric current to attract the beads 301 to the marker 352. The electric potential source 360 can a voltage with a positive electrode 360a and a negative electrode 360b in the solution 356. The electric potential from source 360 orients the beads 301, and the electric potential from source 358 causes the body or body of the marker to attract the beads 301 to the surface of the marker 352. As shown in FIG. 3B, the reflective portions 306 of the beads 301 are oriented toward the marker 352 when the beads are adhered to the surface of the marker. An adhesive 354 secures the beads to the marker in this orientation. The result is that the beads 301 are adhered to the surface of the marker 352 with the reflective portions near the surface of the marker and the transparent portions 304 facing away from the surface of the marker. This forms a reflective coating on the marker 352. More beads can be placed in the solution until the marker 352 is uniformly coated with the beads 301.

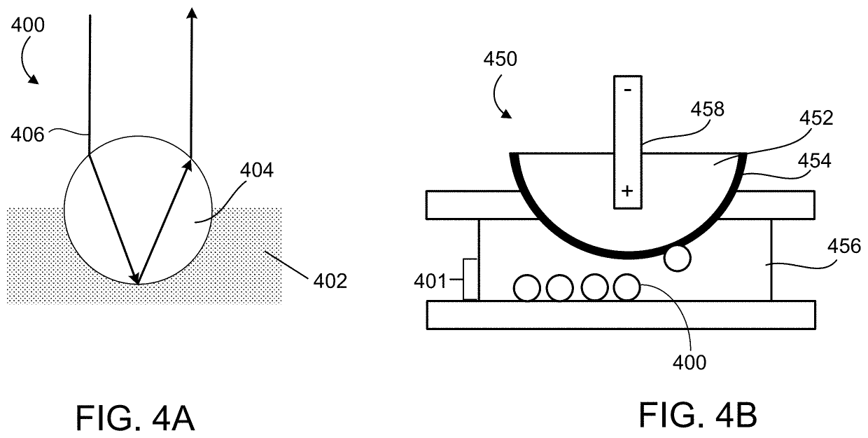

FIGS. 4A and 4B show an example of a bead 400 and a process 450 for coating a marker with beads 401 included the bead 400. The bead 400 has an entirely clear portion 404 and does not include a reflective coating or portion, in contrast to bead 300. The bead 400 is disposed in a medium 402 including reflective particulates (e.g., flakes). The flakes can include aluminum, silver or a similar such reflective material. For example, the spherical surface of the bead is coated with a liquid adhesive including the reflective particulate. Generally, the flakes do not need to be uniform in size or shape. The flakes of the particulate solution or adhesive are generally much smaller than the bead 400 (e.g., 10:1 ratio or similar). The flakes cause the bead 400 to be retroreflective such that an electromagnetic wave 406 is reflected in a parallel but opposite vector.

In FIG. 4B, the beads 401 (e.g., including bead 400) are attached to the marker 452. The orientation of the beads 401 does not need to be controlled. The beads 401 are placed in a solution 456, such as water. The beads 401 have no reflective coating. An electric potential from source 458 is applied to the marker 452 to attract the beads 401 to the surface of the marker. The beads 400 include a material that allows the beads 400 to be electromagnetically attracted to the body of the marker. For example, the material of the beads 400 can be charged to a given potential so that they can be attracted to the body or substrate when a potential is applied to the marker body.

Generally, the marker 452 includes a body or substrate to which the beads 401 are adhered. The body or substrate can include most any material that is configured to receive the adhesive layer 454 and the beads 401. The beads 401 are attracted to the surface of the marker 452 (e.g., using an electric potential) and cured in place. The adhesive 454 includes the particulate material 402. The result is that the beads 401 are uniformly disposed on the curved surface of the marker 452. The adhesive 454 provides the reflective properties of the marker 452.

To ensure proper retro-reflection, the beads 401 are disposed in the medium 402 at a controlled depth. The depth is configured to tune the retro-reflection of the beads, similar to the reflective material described in relation to FIGS. 2A-2C. For example, if the bead 400 is further submerged in the medium 402, the reflective properties will be different (e.g., include greater retro-reflective property) than if the bead is less disposed in the medium. Generally bead 400 is approximately halfway disposed in the medium 402. However, other depths can be used to increase or decrease the luminosity of the bead 400 for tracking purposes.

Generally, the beads 400 can be disposed in the medium at depths other than 50% as previously described. There can be some tolerance on this depth, and a desired performance can be obtained if the average depth of the beads across a visible surface is approximately 50% submersion into the medium. Depth is controlled by applying a layer of the medium 402 at a thickness approximately equal to half the bead height on the spherical surface that has already been coated with a cured layer of the medium. The beads 400 are then applied to the medium 402 while the medium is still in a liquid state. The beads 400 sink and subsequently cure in place at the desired depth. Generally, desired submersion depth can vary from 50% depending on the particular medium and bead materials used.

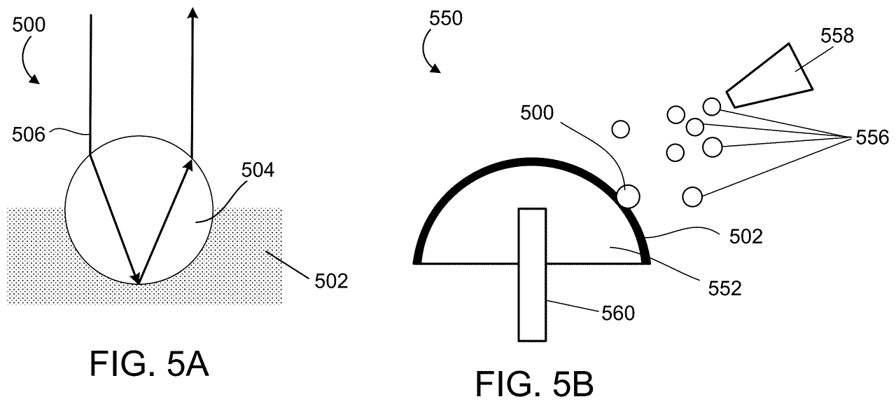

FIGS. 5A and 5B show a bead 500 and a process 550 for applying the bead to a marker 552. In this process 550, a pneumatic process is used to apply the beads 556 (which can include bead 500) on to the marker 552. The process 550 does not require application of a potential to the marker 552. The orientation of the beads 556 does not need to be controlled. Rather, the beads 552 can be in an orientation on the surface of the marker 552.

The bead 500 includes a glass sphere with a clear portion 504 (similar to bead 400). Generally, as with bead 400, the bead 500 is configured to be disposed in a medium 502 which includes reflective particulates. Medium 502 can be the same or similar to medium 402. Medium 502 causes retro-reflection of electromagnetic signal 506.

Generally, the spherical surface of the marker 552 is coated with the material 502, which includes an adhesive containing the reflective particulate. Beads 556 are applied to all surfaces of the marker 552 using a low pressure pneumatic application. The spherical surface of the marker 552 is rotated for even coverage (e.g., using a control mechanism 560). A pressure is selected to achieve a bead acceleration that does not rotate or translate the bead once it has contacted the adhesive medium 502. This prevents smearing the reflective medium away from the surface of the marker 552 onto the beads 556 to avoid obstructing the clear outer surface of the beads. The pressure is applied using a low-pressure pneumatic delivery system 558. A low pressure pneumatic delivery can include a gravity-based delivery.

Generally, the system pneumatic delivery system 558 causes the beads 556 to be about halfway submerged in the adhesive material 502. The pressure of the system 558 can be tuned to control how deep into the adhesive the beads 556 are placed. This can tune the retro-reflective properties of the marker 556, as previously described. For example, the material properties of the medium which the beads are embedded can also influence the amount of submersion of the bead and influence the retroreflective properties.

For each of the beads and processes previously and subsequently described, the bead dispersion pattern and application velocity are controlled to control uniformity of the reflection of the marker. In some implementations, a desired uniformity is function of pixel density of sensor or a curvature of surface. Thus, the desired uniformity is achieved using the processes and beads described herein. To test the uniformity of a marker, a change in reflection intensity is measured over the surface of the marker. Generally, a weighted peak intensity is obtained for the marker. The marker is rotated, and changes in position measurement of peak intensity are measured. A lower change in peak intensity indicates a higher uniformity of the reflective coating.

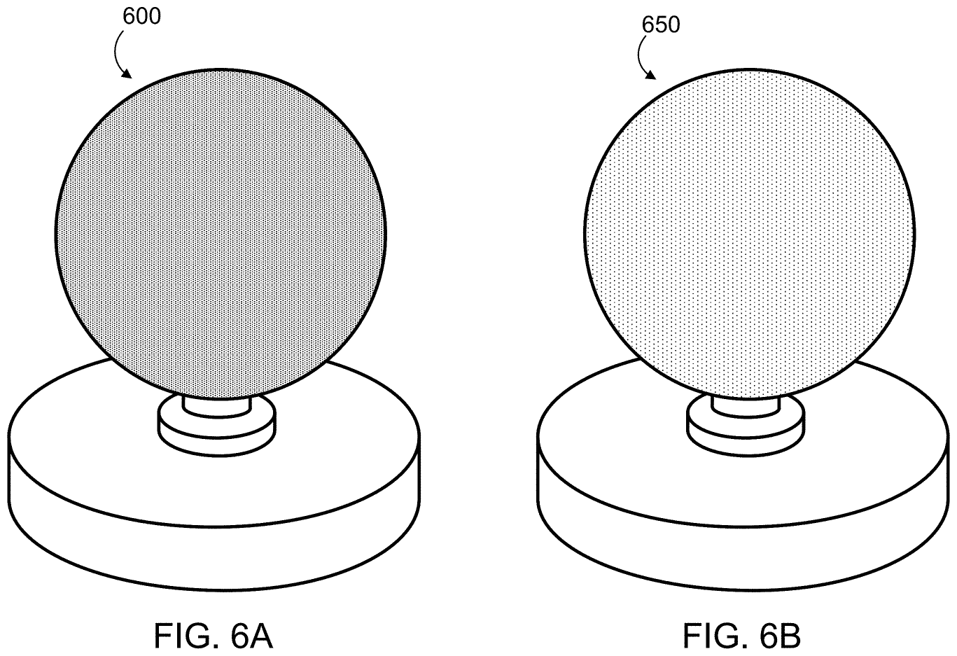

FIGS. 6A and 6B show images of an example marker in a non-illuminated state 600 and an illuminated state 650. The marker can be rotated so that all of the surface is visible over time. The peak intensity of the reflections can be measured to test uniformity. For example, image 650 corresponds to a camera flash on the marker. The camera flash occurs from a location near the image sensor that captures the images of states 600, 650.

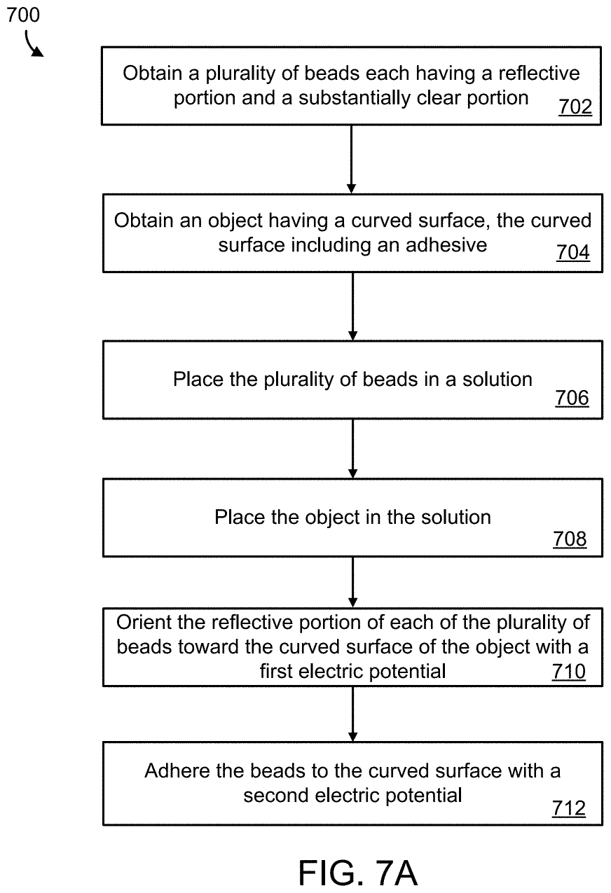

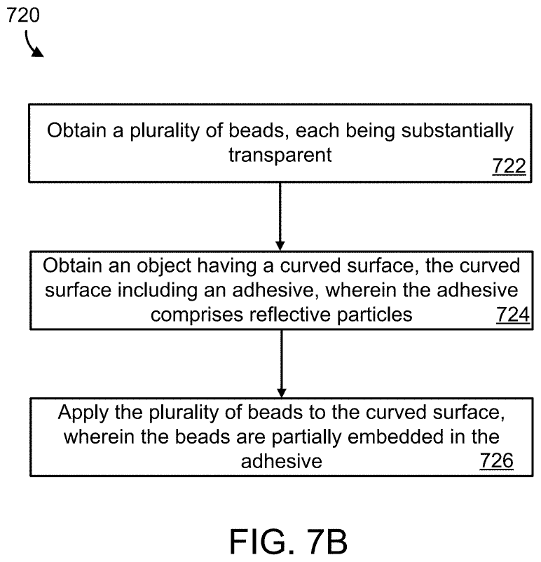

FIGS. 7A and 7B show flow diagrams each illustrating an example process for producing a marker for an optical tracking system. The process 700 of FIG. 7A includes obtaining (702) a plurality of beads each having a reflective portion and a substantially clear portion. The process 700 includes obtaining (704) an object having a curved surface, the curved surface including an adhesive. The process 700 includes placing (706) the plurality of beads in a solution. The process 700 includes placing (708) the object in the solution. The process 700 includes applying (710) a first electric potential to the solution, wherein the electric potential orients the reflective portion of each of the plurality of beads toward the curved surface of the object. The process 700 includes applying (712) a second electric potential to the object to attract the beads of the plurality to the curved surface, wherein the adhesive on the curved surface holds each of the beads on the curved surface of the object. The reflective portion of each bead is oriented toward the curved surface. The substantially clear portion of each bead is oriented away from the curved surface.

In some implementations, each bead includes a bead body that is configured to be a dipole. Generally, a first pole of the dipole is positioned at the reflective portion of the bead. A second pole of the dipole is positioned at the substantially clear portion of the bead. In some implementations, each bead comprises barium titanate. In some implementations, the plurality of beads coat the curved surface of the object to cause a reflective uniformity a predetermined threshold. In some implementations, the object that forms the marker is a sphere. In some implementations, each bead is a sphere. The reflective portion of each bead is approximately a first hemisphere of the sphere, and the substantially clear portion is a second hemisphere of the sphere. In some implementations, the reflective portion comprises aluminum, silver, or a combination thereof.

FIG. 7B shows process 720 for producing a reflective marker for an optical tracking system. The process 700 includes obtaining (722) a plurality of beads each being substantially transparent. The process 720 includes obtaining (724) an object having a curved surface. Generally, the curved surface includes an adhesive. The adhesive includes reflective particles. The process 720 includes applying (726) the plurality of beads to the curved surface, wherein the beads are partially embedded in the adhesive. The process 720 includes applying the plurality of beads to the curved surface using a low-pressure pneumatic application of the beads to the adhesive. In some implementations, the reflective particles in the adhesive comprise silver, aluminum, or a combination thereof. In some implementations, a size of the reflective particles is about one-tenth of a size of the beads. In some implementations, applying the plurality of beads to the curved surface comprises embedding about one half of each of the beads in the adhesive.

In some implementations, applying the plurality of beads to the curved surface comprises applying an electric potential to the object. In some implementations, the electric potential attracts the beads to adhere to the adhesive.

A number of implementations of the invention have been described. Nevertheless, it will be understood that various modifications may be made without departing from the spirit and scope of the invention. For example, the position of markers and tracked object can change with time. The computing device may be configured to automatically detect which bright spots in an image identified as marker reflections at a first time and at first positions correspond to marker reflections that are identified at a second time and at second positions. Accordingly, other implementations are within the scope of the following claims.

Some implementations of subject matter and operations described in this specification can be implemented in digital electronic circuitry, or in computer software, firmware, or hardware, including the structures disclosed in this specification and their structural equivalents, or in combinations of one or more of them. For example, in some implementations, tracking system 100 and the computing system 110 can be implemented using digital electronic circuitry, or in computer software, firmware, or hardware, or in combinations of one or more of them.

Some implementations described in this specification can be implemented as one or more groups or modules of digital electronic circuitry, computer software, firmware, or hardware, or in combinations of one or more of them. Although different modules can be used, each module need not be distinct, and multiple modules can be implemented on the same digital electronic circuitry, computer software, firmware, or hardware, or combination thereof.

Some implementations described in this specification can be implemented as one or more computer programs, i.e., one or more modules of computer program instructions, encoded on computer storage medium for execution by, or to control the operation of, data processing apparatus. For example, a computing system (such as computing system 110) can be used in the tracking system to control operation of the emitter and to process the images captured by the image sensor. In some implementations, a computing system can be used to control application of voltages during application of the beads to the marker body or to control pneumatic application of the beads to the marker body. A computer storage medium can be, or can be included in, a computer-readable storage device, a computer-readable storage substrate, a random or serial access memory array or device, or a combination of one or more of them. Moreover, while a computer storage medium is not a propagated signal, a computer storage medium can be a source or destination of computer program instructions encoded in an artificially generated propagated signal. The computer storage medium can also be, or be included in, one or more separate physical components or media (e.g., multiple CDs, disks, or other storage devices).

The term “data processing apparatus” encompasses all kinds of apparatus, devices, and machines for processing data, including by way of example a programmable processor, a computer, a system on a chip, or multiple ones, or combinations, of the foregoing. In some implementations, computing system 110 includes a data processing apparatus as described herein. The apparatus can include special purpose logic circuitry, e.g., an FPGA (field programmable gate array) or an ASIC (application specific integrated circuit). The apparatus can also include, in addition to hardware, code that creates an execution environment for the computer program in question, e.g., code that constitutes processor firmware, a protocol stack, a database management system, an operating system, a cross-platform runtime environment, a virtual machine, or a combination of one or more of them. The apparatus and execution environment can realize various different computing model infrastructures, such as web services, distributed computing and grid computing infrastructures.

A computer program (also known as a program, software, software application, script, or code) can be written in any form of programming language, including compiled or interpreted languages, declarative or procedural languages. A computer program may, but need not, correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed for execution on one computer or on multiple computers that are located at one site or distributed across multiple sites and interconnected by a communication network.

Some of the processes and logic flows described in this specification can be performed by one or more programmable processors executing one or more computer programs to perform actions by operating on input data and generating output. The processes and logic flows can also be performed by, and apparatus can be implemented as, special purpose logic circuitry, e.g., an FPGA (field programmable gate array) or an ASIC (application specific integrated circuit).

Processors suitable for the execution of a computer program include, by way of example, both general and special purpose microprocessors, and processors of any kind of digital computer. Generally, a processor will receive instructions and data from a read only memory or a random access memory or both. A computer includes a processor for performing actions in accordance with instructions and one or more memory devices for storing instructions and data. A computer may also include, or be operatively coupled to receive data from or transfer data to, or both, one or more mass storage devices for storing data, e.g., magnetic, magneto optical disks, or optical disks. However, a computer need not have such devices. Devices suitable for storing computer program instructions and data include all forms of non-volatile memory, media and memory devices, including by way of example semiconductor memory devices (e.g., EPROM, EEPROM, flash memory devices, and others), magnetic disks (e.g., internal hard disks, removable disks, and others), magneto optical disks, and CD-ROM and DVD-ROM disks. The processor and the memory can be supplemented by, or incorporated in, special purpose logic circuitry.

To provide for interaction with a user, operations can be implemented on a computer having a display device (e.g., a monitor, or another type of display device) for displaying information to the user and a keyboard and a pointing device (e.g., a mouse, a trackball, a tablet, a touch sensitive screen, or another type of pointing device) by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; for example, by sending web pages to a web browser on a user's client device in response to requests received from the web browser.

A computer system may include a single computing device, or multiple computers that operate in proximity or generally remote from each other and typically interact through a communication network. Examples of communication networks include a local area network (“LAN”) and a wide area network (“WAN”), an inter-network (e.g., the Internet), a network comprising a satellite link, and peer-to-peer networks (e.g., ad hoc peer-to-peer networks). A relationship of client and server may arise by virtue of computer programs running on the respective computers and having a client-server relationship to each other.

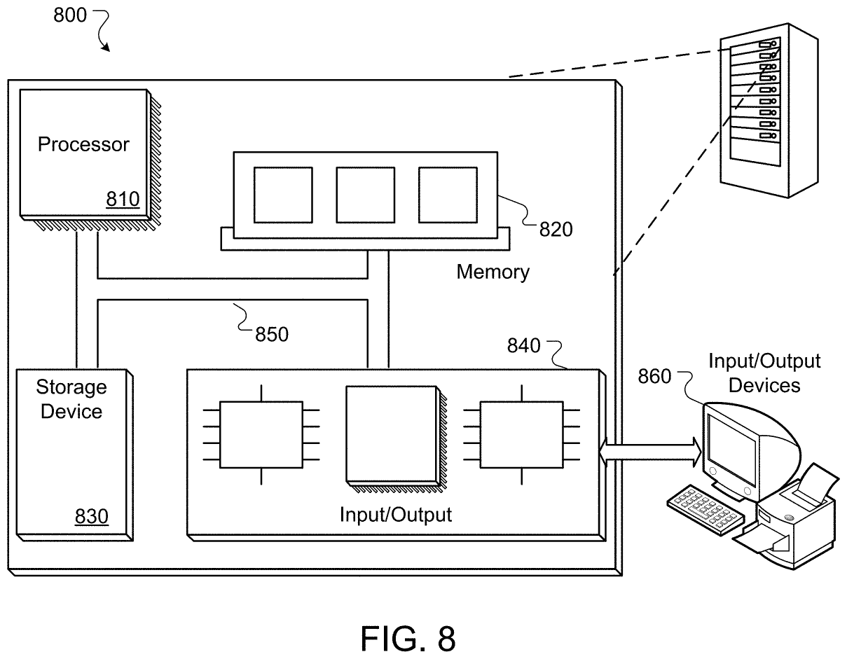

FIG. 8 shows an example computer system 800 (e.g., similar to or including computing system 110) that includes a processor 810, a memory 820, a storage device 830 and an input/output device 840. Each of the components 810, 820, 830 and 840 can be interconnected, for example, by a system bus 850. The processor 810 is capable of processing instructions for execution within the system 800. In some implementations, the processor 810 is a single-threaded processor, a multi-threaded processor, or another type of processor. The processor 810 is capable of processing instructions stored in the memory 820 or on the storage device 830. The memory 820 and the storage device 830 can store information within the system 800.

The input/output device 840 provides input/output operations for the system 800. In some implementations, the input/output device 840 can include one or more of a network interface device, e.g., an Ethernet card, a serial communication device, e.g., an RS-232 port, and/or a wireless interface device, e.g., an 802.11 card, a 3G wireless modem, a 4G wireless modem, a 8G wireless modem, etc. In some implementations, the input/output device can include driver devices configured to receive input data and send output data to other input/output devices, e.g., keyboard, printer and display devices 860. In some implementations, mobile computing devices, mobile communication devices, and other devices can be used.

While this specification contains many details, these should not be construed as limitations on the scope of what may be claimed, but rather as descriptions of features specific to particular examples. Certain features that are described in this specification in the context of separate implementations can also be combined. Conversely, various features that are described in the context of a single implementation can also be implemented in multiple embodiments separately or in any suitable sub-combination.

Thus, specific embodiments of the optical tracking system and retro-reflective marker and methods for using the optical tracking system to track retro-reflective markers have been disclosed. It should be apparent, however, to those skilled in the art that many more modifications besides those already described are possible without departing from the inventive concepts herein. The inventive subject matter, therefore, is not to be restricted except in the spirit of the disclosure. Moreover, in interpreting the disclosure, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, a limited number of the exemplary methods and materials are described herein.

What is claimed is:

1. A marker for an optical tracking system, the marker comprising:
an object having a curved surface;
a plurality of beads adhered to the curved surface with an adhesive, the beads of the plurality configured for being retroreflectors, wherein a bead of the plurality of beads comprises:
a first portion having a reflective surface; and
a second portion that is substantially transparent;
wherein the first portion of the bead is oriented toward the curved surface of the object; and
wherein the second portion of the bead is oriented away from the curved surface of the object.

2. The marker of claim 1, wherein the bead of the plurality of beads further comprises:
a bead body that is configured to be a dipole, wherein a first pole of the dipole is positioned at the first portion of the bead, and wherein a second pole of the dipole is positioned at the second portion of the bead.

3. The marker of claim 1, wherein the bead comprises a polarizable material that is at least substantially transparent in a near infrared (NIR) band with refractive index that is greater than 1.6.

4. The marker of claim 3, wherein the bead comprises a material selected from a group of materials including Barium Titanate.

5. The marker of claim 1, wherein the object is a spheriod.

6. The marker of claim 1, wherein the bead is a spheriod, and wherein the first portion is approximately a first hemisphere of the spheriod, and wherein the second portion is a second hemisphere of the spheriod.

7. The marker of claim 1, wherein the reflective surface comprises aluminum, silver, or a combination thereof.

8. A method for producing a reflective marker for an optical tracking system, the method comprising:
obtaining a plurality of beads each having a reflective portion and a substantially clear portion;
obtaining an object having a curved surface, the curved surface including an adhesive;
placing the plurality of beads in a solution;
placing the object in the solution;
applying a first electric potential to the solution, wherein the first electric potential orients the reflective portion of each of the plurality of beads toward the curved surface of the object; and
applying a second electric potential to the object to attract the beads of the plurality to the curved surface, wherein the adhesive on the curved surface holds each of the beads on the curved surface of the object, wherein the reflective portion of each of the beads is oriented toward the curved surface, and wherein the substantially clear portion is oriented away from the curved surface.

9. The method of claim 8, wherein each bead further comprises:
a bead body that is configured to be a dipole, wherein a first pole of the dipole is positioned at the reflective portion of the bead, and wherein a second pole of the dipole is positioned at the substantially clear portion of the bead.

10. The method of claim 8, wherein the bead comprises a polarizable material that is at least substantially transparent in a near infrared (NIR) band with refractive index that is greater than 1.6.

11. The method of claim 10, wherein the bead comprises a material selected from a group of materials including Barium Titanate.

12. The method of claim 8, wherein the object is a spheriod.

13. The method of claim 8, wherein the bead is a spheriod, and wherein the reflective portion is approximately a first hemisphere of the spheriod, and wherein the substantially clear portion is a second hemisphere of the spheriod.

14. The method of claim 8, wherein the reflective portion comprises aluminum, silver, an alloy of aluminum or silver, or a combination thereof.

15. A method for producing a reflective marker for an optical tracking system, the method comprising:
obtaining a plurality of beads each being substantially transparent;
obtaining an object having a curved surface, the curved surface including an adhesive, wherein the adhesive comprises reflective particles; and
applying the plurality of beads to the curved surface, wherein the beads are partially embedded in the adhesive.

16. The method of claim 15, wherein applying the plurality of beads to the curved surface a low-pressure pneumatic application of the beads to the adhesive.

17. The method of claim 15, wherein the reflective particles in the adhesive comprise silver, aluminum, or a combination thereof.

18. The method of claim 15, wherein a size of the reflective particles is approximately one-tenth or less of a size of the beads.

19. The method of claim 15, wherein applying the plurality of beads to the curved surface comprises embedding about one half of each of the beads in the adhesive.

20. The method of claim 15, wherein applying the plurality of beads to the curved surface comprises applying an electric potential to the object, wherein the electric potential attracts the beads to adhere to the adhesive.
